(19) United States
(12) Patent Application Publication
(10) Pub. No.: US 2007/0075159 A1
(43) Pub. Date: Apr. 5, 2007

(54) Air freshener

(76) Inventor: Jeng-Hsi Lin, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/240,500

(22) Filed: Oct. 3, 2005

(51) Int. Cl. A61L 9/04

(52) U.S. Cl. 239/60; 239/57

(57) ABSTRACT

An air freshener has a casing, a solid aromatic, a cover, an air passage and a fastening device. The casing has a cavity and an air inlet. The aromatic solid is mounted securely in the cavity. The cover is mounted on the casing and has an air vent. The air passage is between the cover and the casing and communicates with the cavity, the air vent and the air inlet. Air flows into the air freshener through the air inlet, passes through the air passage and leaves through the air outlet. When the air passes through the air passage, the fragrance of the aromatic solid is mixed with and leaves with the air discharged through the air vent.

### BACKGROUND OF THE INVENTION

1. Field of the Invention

The present invention relates to an air freshener, especially to an air freshener that contains an aromatic solid for vehicles, houses or offices.

2. Description of the Prior Arts

Air fresheners are mounted in vehicles, houses or offices to remove or mask unpleasant odors in the air. With reference to FIG. 4, a conventional air freshener for a vehicle comprises an outer casing (90), an inner container (91), aromatic liquid (92), a wick (93), a top cover (94) and a clip (95). The outer casing (90) has a top opening. The inner container (91) is mounted in the outer casing (90), extends out from the top opening of the outer casing (90) and has a top opening. The aromatic liquid (92) is held in the inner container (91), has a fragrance to make the air smell good and may contain essential oil to settle one's mind, to keep up one's spirit, etc. The wick (93) is mounted in the aromatic liquid (92) in the inner container (91) and protrudes from the top opening of the inner container (91) to emit the fragrance of the aromatic liquid (92). The top cover (94) is attached to the top opening of the outer casing (90) and has multiple elongated through holes (941). The elongated through holes (941) are formed through the top cover (94) to emit the fragrance of the aromatic liquid (92). The clip (95) is attached to the outer casing (90) to clamp vanes in an air outlet of an air condition in vehicles, homes or offices. The elongated through holes (941) must face upward to keep the aromatic liquid (92) from leaking out of the air freshener. However, the vanes in the air outlet pivot to distribute cooled air being discharged from the air conditioner. When the vanes pivot, the aromatic liquid (92) may leak because the direction in which the elongated through holes (941) face is changed and may not face upward. Furthermore, vibration of the vehicle when driven may shake the conventional air freshener and make the aromatic liquid (92) spill out easily.

To overcome the shortcomings, the present invention provides an air freshener that contains solid aromatic to mitigate or obviate the aforementioned problems.

### SUMMARY OF THE INVENTION

The main objective of the present invention is to provide an air freshener that contains an aromatic solid to keep the aromatic substance from being shaken out by vibration. The air freshener has a casing, an aromatic solid, a cover, an air passage and a fastening device. The casing has a cavity and an air inlet. The aromatic solid is mounted securely in the cavity. The cover is mounted on the casing and has an air vent. The air passage is between the cover and the casing and communicates with the cavity, the air vent and the air inlet. Air flows into the air freshener though the air inlet, passes through the air passage and leaves through the air vent. When the air passes through the air passage, the fragrance of the aromatic solid is mixed with and leaves with the air discharged through the air vent.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an air freshener in accordance with the present invention;

FIG. 2 is an exploded perspective view of the air freshener in FIG. 1;

FIG. 3 is a side view in partial section of the air freshener in FIG. 1; and

FIG. 4 is a side view in partial section of a conventional air freshener in accordance with the prior art.

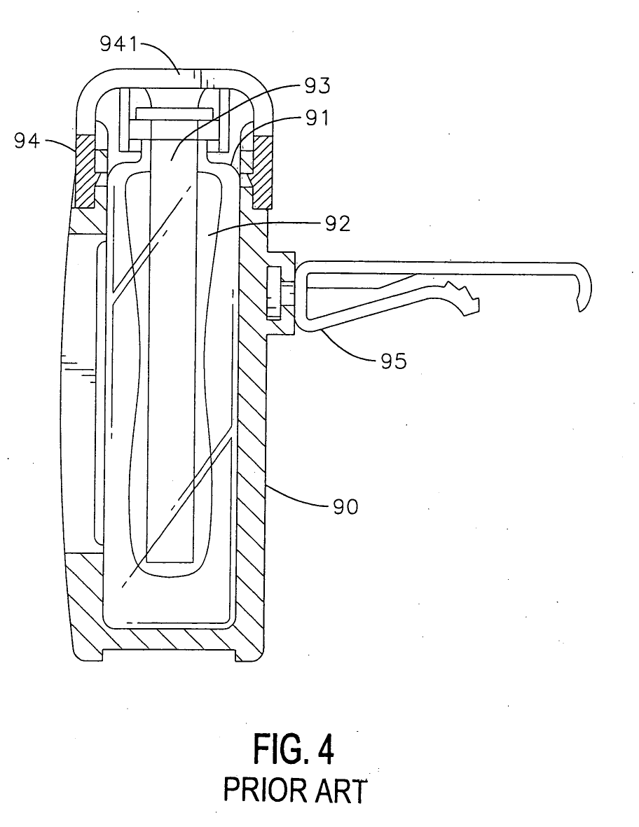

### DETAILED DESCRIPTION OF THE PREFERRED EMBODIMENTS

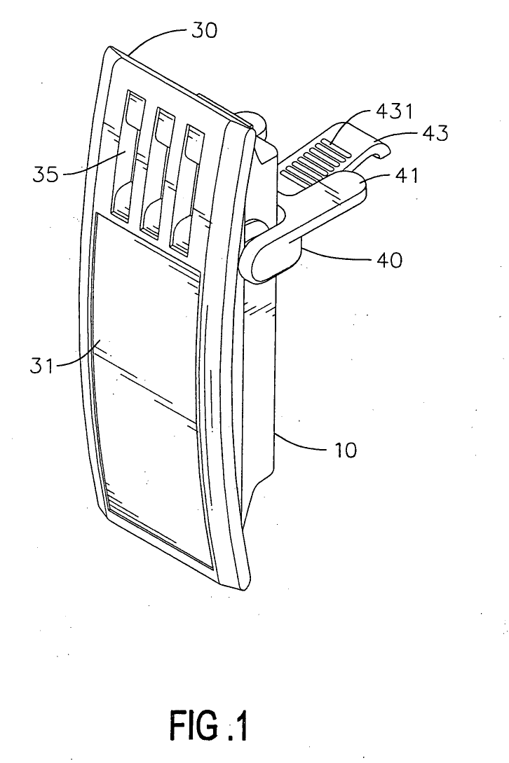

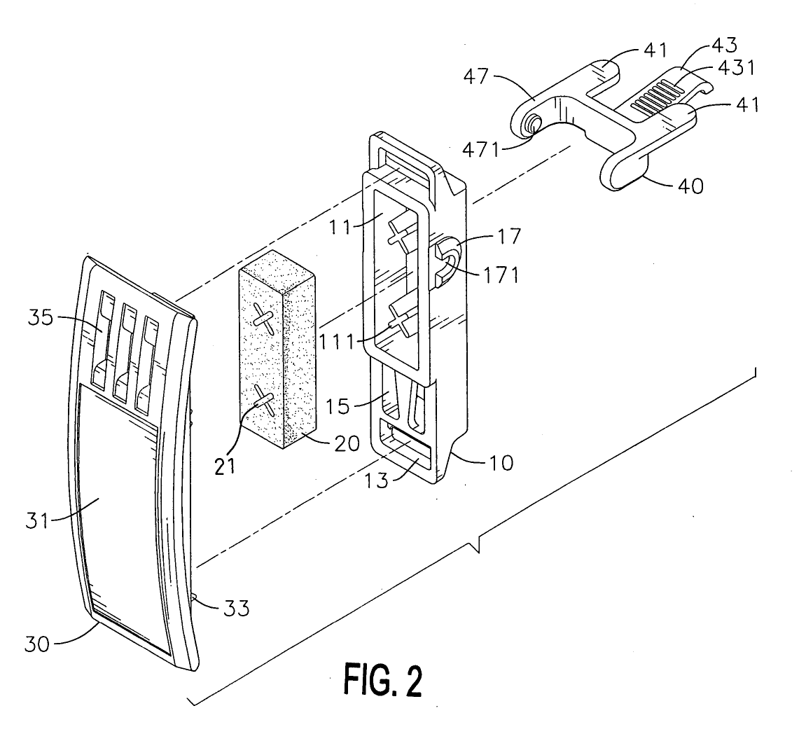

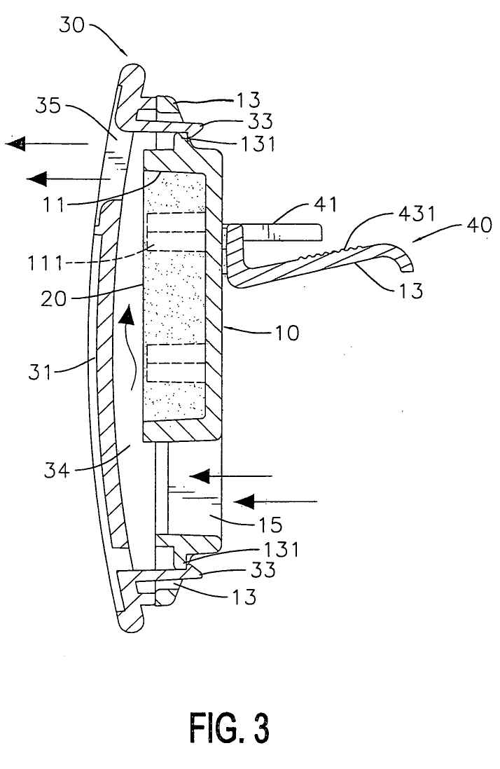

With reference to FIGS. 1, 2 and 3, an air freshener in accordance with the present invention comprises a casing (10), an aromatic solid (20), a cover (30), an air passage (34) and a fastening device (40).

The casing (10) has an inner surface, two ends, two sides, a cavity (11), at least one air inlet (15), two optional ears (13), two optional lips (131) and two optional protrusions (17). The cavity (11) is formed in the inner surface of the casing (10), has an opening and may have at least one positioning protrusion (111). The at least one positioning protrusion (111) is formed in the cavity (11) and may be cross-shaped, arc-shaped, polygon-shaped, etc. The at least one air inlet (15) is formed in the casing (10) and communicates with the cavity (11) in the casing (10). The ears (13) are formed respectively on the ends of the casing (10). The lips (131) are formed respectively on the ends of the casing (10) near the ears (13). The protrusions (17) are C-shaped, are formed respectively on the sides of the casing (10), and each protrusion (17) has an opening (171). The opening (171) in the protrusion (17) faces the inner surface of the casing (10).

The aromatic solid (20) is mounted securely in the cavity (11) of the casing (10) and may have at least one positioning hole (21). The at least one positioning hole (21) is formed through the aromatic solid (20) and corresponds to and engages the at least one positioning protrusion (111) in the cavity (11).

The cover (30) is mounted on the inner surface of the casing (10) and has an outer surface, an inner surface, two ends, at least one air vent (35), two optional hooks (33) and an optional recess (31). The air vent (35) is formed through the cover (30). The hooks (33) are formed on the inner surface respectively near the two ends of the cover (30), extend respectively through the ears (13) on the casing (10) and correspond respectively to and hook the lips (131) on the casing (10). The recess (31) is formed in the outer surface of the cover (30) to mount marks, labels or instructions.

The air passage (34) is formed between the inner surface of the cover (30) and the inner surface of the casing (10) and communicates with the cavity (11) in the casing (10), the air inlet (15) in the casing (10) and the air vent (35) in the cover (30).

The fastening device (40) is attached to the casing (10), may be mounted pivotally in the protrusions (17) on the casing (10) and may have a rear surface, two mounting wings (47), two stationary clamp arms (41) and a resilient clamp arm (43). The mounting wings (47) are formed on the fastening device (40) and corresponds respectively to and are mounted pivotally on the protrusions (17) on the casing (10). Each mounting wing (47) has a pivoting protrusion (471) formed on the mounting wing (47) and mounted pivotally in the opening (171) in one of the protrusions (17). The stationary clamp arms (41) are formed on the rear surface of the fastening device (40) and extend away from the mounting wings (47). The resilient clamp arm (43) is formed on the rear surface of the fastening device (40), extends away from the mounting wings (47) and has multiple optional ribs (431). The ribs (431) are formed on the resilient clamp arm (43). The stationary and resilient clamp arms (41, 43) clamp the air freshener in position in vehicles, houses and offices.

Air enters the air freshener through the air inlet (15) in the casing (10), passes through the air passage (34) between the cover (30) and the casing (10) and leaves through the air vent (35) in the cover (30). When the air passes through the air passage (34), the fragrance of the aromatic solid (20) mixes with and leaves with the air.

The air freshener as described has numerous advantages. The aromatic solid (20) does not leak out of the air freshener no matter how the air freshener is shaken. With the air passage (34), the fragrance of the aromatic solid (20) easily dispenses from the air freshener.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An air freshener comprising
a casing having
an inner surface;
two ends;
two sides;
a cavity formed in the inner surface of the casing having an opening; and
at least one air inlet formed in the casing and communicating with the cavity of the casing;
an aromatic solid mounted securely in the cavity of the casing;
a cover mounted on the inner surface of the casing and having
an outer surface;
an inner surface;
two ends; and
at least one air vent formed through the cover;
an air passage formed between the inner surface of the cover and the inner surface of the casing and communicating with the cavity in the casing, the air inlet in the casing and the air vent in the cover; and
a fastening device attached to the casing.

2. The air freshener as claimed in claim 1, wherein the casing has
two ears formed respectively on the ends of the casing; and
two lips formed respectively on the ends of the casing near the ears; and
the cover has two hooks formed on the inner surface respectively near the two ends of the cover, extending respectively through the ears on the casing and corresponding respectively to and hooking the lips on the casing.

3. The air freshener as claimed in claim 1, wherein
the casing has two protrusions being C-shaped, formed respectively on the sides of the casing, and each protrusion has an opening facing the inner surface of the casing; and
the fastening device is mounted pivotally in the protrusions on the casing and has
a rear surface;
two mounting wings formed on the fastening device and corresponding respectively to and mounted pivotally on the protrusions on the casing, and each mounting wing having a pivoting protrusion formed on the mounting wing and mounted pivotally in the opening in one of the protrusions;
two stationary clamp arms formed on the rear surface of the fastening device and extending away from the mounting wings; and
a resilient clamp arm formed on the rear surface of the fastening device and extending away from the mounting wings.

4. The air freshener as claimed in claim 2, wherein
the casing has two protrusions being C-shaped, formed respectively on the sides of the casing, and each protrusion has an opening facing the inner surface of the casing; and
the fastening device is mounted pivotally in the protrusions on the casing and has
a rear surface;
two mounting wings formed on the fastening device and corresponding respectively to and mounted pivotally on the protrusions on the casing, and each mounting wing having a pivoting protrusion formed on the mounting wing and mounted pivotally in the opening in one of the protrusions;
two stationary clamp arms formed on the rear surface of the fastening device and extending away from the mounting wings; and
a resilient clamp arm formed on the rear surface of the fastening device and extending away from the mounting wings.

5. The air freshener as claimed in claim 3, wherein the resilient clamp arm has multiple ribs formed on the resilient clamp arm.

6. The air freshener as claimed in claim 4, wherein the resilient clamp arm has multiple ribs formed on the resilient clamp arm.

7. The air freshener as claimed in claim 1, wherein
the cavity in the casing has at least one positioning protrusion formed in the cavity; and
the aromatic solid has at least one positioning hole formed through the aromatic solid and corresponding to and engaging the at least one positioning protrusion in the cavity.

8. The air freshener as claimed in claim 6, wherein
the cavity of the casing has at least one positioning protrusion formed in the cavity; and
the aromatic solid has at least one positioning hole formed through the aromatic solid and corresponding to and engaging the at least one positioning protrusion in the cavity.

9. The air freshener as claimed in claim 7, wherein the at least one positioning protrusion in the cavity is cross-shaped.

10. The air freshener as claimed in claim 8, wherein the at least one positioning protrusion in the cavity is cross-shaped.

11. The air freshener as claimed in claim 1, wherein the cover has a recess formed in the outer surface of the cover.
